(19) United States
(12) Patent Application Publication
(10) Pub. No.: US 2023/0172904 A1
(43) Pub. Date: Jun. 8, 2023

(54) COMBINATION OF AN ANTIALLERGIC AGENT WITH MUSCARINIC ANTAGONIST AND/OR DOPAMINERGIC AGONIST FOR USE IN PREVENTING/STOPPING OF AXIAL MYOPIA IN HUMAN

(71) Applicant: Enzo Maria D'AMBROSIO, Manduria (IT)

(72) Inventor: Enzo Maria D'AMBROSIO, Manduria (IT)

(21) Appl. No.: 18/096,165

(22) Filed: Jan. 12, 2023

(30) Foreign Application Priority Data
Feb. 19, 2016 (IT) 102016000017487

(51) Int. Cl. A61K 31/4035; A61K 47/02; A61K 9/00

(52) U.S. Cl. CPC A61K 31/4035; A61K 47/02; A61K 9/0048

(57) ABSTRACT

One of the major issues against the use of muscarinic antagonists or dopamine agonist eyedrops for the controlling of eye growth and prevention of myopia is the unacceptable rate of iatrogenic conjunctivitis or dermatitis. This invention relates to the association of those active principles with an antiallergic component. In alternative the ophthalmic use of a molecule that simultaneously has an antimuscarinic and/or dopaminergic action along with an antihistaminic function.

This application is a national stage application under 35 U.S.C. § 371 of PCT Application No. PCT/EP2017/053619, filed Feb. 17, 2017, which claims priority of Italy Patent Application No. 102016000017487, filed Feb. 19, 2016, which are hereby incorporated by reference in their entirety.

### FIELD OF THE INVENTION

The present invention relates to the field of combination of pharmaceutical active ingredient or multifunctional active principles for use in preventing/stopping of myopia in human.

### STATE OF THE ART

Myopia could be defined as the need for an eye of a negative lens to focus an image on the retinal plane. A number of subgroup of myopia exists, the most diffuse is the axial myopia, due to an excessive elongation of the ocular bulb. In most of western child the growth of the eye proceeds constantly, and the eye physiologically maintains a little farsightedness, but in some, mainly between 6-14 years, the ocular elongation is faster and results in nearsightedness. The underlining biological process is still poorly understood, genetic and ambiental factors contribute to its development. It's known that myopia is three times more prevalent in Asia than in western countries, some studies relate the development of the condition to the number of hours of indoor study or, inversely, to the time spent outside. In experimental model, chicks or small mammalian, excessive eye growth could be elicited by lid suture or degradation of the image using translucent lenses (form deprivation myopia or FDM) or, interestingly by the apposition of negative lenses (lens induced myopia or LIM). Are also available breeds of mice with a spontaneous abnormal eye growth.

Many do not consider myopia as a disease, because in most of cases is acceptably corrected by the means of glasses or contact lenses, but it could be intended as a disfunction of the eye growth. Moreover contact lens wearers are subject to allergic or foreign body reactions or more serious corneal infection that can cause loss of vision. Furthermore, a myopic eye is more subject to retinal degeneration and retinal tears that can lead to retinal detachment, glaucoma, myopic foveoschisis and macular hole, all causes of blindness. So effective therapy for preventing myopia is needed not only for esthetic finalities but also to prevent blinding disease in adult age.

Since the studies of R. Bedrossian (Ophthalmology, May 1979, Vol 86, pp. 713-717), a growing body of evidence shows that the blocking of M1 muscarinic receptor in the eye halt the axial elongation of the eye both in experimental models (Form deprivation myopia or lens induced myopia) and in human.

In the last 3 decades a number of studies explored the risks and benefits of Atropine eyedrops at various concentration or in association with other devices, like contact lenses or multifocal glasses, in order to find an effective formulation for Myopia prevention. These are analyzed in a Cochrane meta analysis (Walline J J et al, Interventions to slow progression of myopia in children. Cochrane Database of Systematic Reviews 2011, Issue 12. Art. No.: CD004916). The better results were obtained from the ATOM (Atropine for Treatment Of Myopia) I and II studies. In the first study, published in 2006, (Chua W. H. et al., Atropine for the Treatment of Childhood Myopia, Ophthalmology 2006; 113:2285-2291) the authors demonstrated, on a placebo controlled large cohort of chinese children, that the bedtime instillation of one drop of 1% Atropine collyrium halted the development of axial myopia in 90% of subjects, by stopping the elongation of the bulb. Since the main factor obstaculating the widespread use of the drug is the vision blurring due to mydriasis, also largely complained in ATOM I study, the same group tried in the second study (Chia A. et al, Atropine for the Treatment of Childhood Myopia: Safety and Efficacy of 0.5%, 0.1%, and 0.01% Doses (Atropine for the Treatment of Myopia 2), Ophthalmology 2012; 119:347-354) to reduce the dose to 0.1%, 0.05% and 0.01% demonstrating a dose dependent effect of atropine that is still acceptable at the lower concentration. By the way a small, around 5%, but still unacceptable number of subject, in a pediatric population, developed ocular redness and itching. This effect was proven triggered by an allergic response of the conjunctiva. (Yoshikawa K., Kalahari S. Contact allergy to atropine and other mydriatic agents; CONTACT DERMATITIS 12(1):56 57, april 2006). The stopping of eye elongation is not mediated by toxic effect on the retina, as postulated in some of the initial in vitro studies, further in vitro analysis and mfERG on human subject did not displayed any alteration on retinal function. (Chia A. et al. Full-field electroretinogram findings in children in the atropine treatment for myopia (ATOM2) study; Documenta Ophthalmologica 126(3) January 2013).

Other muscarinic antagonists were tried, both on experimental model and human, in the attempt to reduce the side effects, in particular mydriasis. Principally pirenzepine was tried (EP 0478694 B1), but the effect correlated anyway to the degree of mydriasis and eye surface sensitization was more than with atropine, thus this way was abandoned.

Further studies, mainly in the last decade of the past century, explored more deeply the role of dopamine (DA) in the process of ocular axial elongation. In experimental models D1 agonists mainly enhanced the ocular elongation while D2 agonists and D1 antagonists block the process. Thus myopia could be explained as an imbalance between the two receptors activity. Results in human are not available and the development of eyedrops based on this pharmaceutical class is difficult because of poor solubility of the active principle at physiological pH. Human studies of Dopaminergic drug in human cannot be found, only experimental data is available, (Feldkaemper M. et al, An updated view on the role of dopamine in myopia, Experimental Eye Research 114 (2013) 106-119) and also dopamine reuptake inhibitors are active against experimental models of myopia induction. Dopamine agonists are nowadays used in Ophthalmology to restore a physiologic intraocular pressure (IOP) in severely compromised eyes, they have little effect on healthy eyes, and showed no risks in chronic use.

It is therefore apparent that one of the major issues against the use of muscarinic antagonists or dopamine agonist eyedrops for the controlling of eye growth and prevention of myopia is the unacceptable rate of iatrogenic conjunctivitis or dermatitis. Aim of the present invention is to provide an ophthalmic treatment for preventing/stopping myopia in children with relief of the observed side effects with muscarinic antagonists or dopamine agonist eyedrops.

### Definition and Abbreviations

AM: Axial myopia
FDM: Form deprivation myopia
LIM: Lens induced myopia
mfERG: multifocal Electroretinogram
DA: Dopamine
IOP: Intraocular pressure

### SUMMARY OF THE INVENTION

The present invention resolves the above problem combining more pharmaceutical active principles in a single formulation wherein an antihistaminic principle is combined with an anticholinergic principle and/or a dopaminergic principle or monoamine reuptake inhibitor; or
a single pharmaceutical active principle having a combined activity as anticholinergic (Antimuscarinic) and antihistaminic, or as anticholinergic, antihistaminic and dopaminergic or monoamine reuptake inhibition;
for use in preventing/stopping axial myopia in children.

Surprisingly the above combination of principle/activity allows the prevention of eye axial elongation without giving rise to drawbacks, like mydriasis and allergic conjunctivitis or dermatitis, observed by the administration of atropine alone.

Further object of the present invention is therefore also an ophthalmic formulation, for use in preventing/stopping axial myopia, comprising:
an antihistaminic principle combined with an anticholinergic principle and/or a dopaminergic principle or monoamine reuptake inhibitor; or
a single pharmaceutical active principle having a combined activity as anticholinergic and antihistaminic, or as anticholinergic, antihistaminic and dopaminergic or monoamine reuptake inhibition (specifically dopamine).

### DETAILED DESCRIPTION OF THE INVENTION

The combination of active principles or the single (antiparkinsonian) principle for use according to the invention are preferably to be administered ocularly, topically or locally to the eye.

According to the invention the composition formulated for ophthalmic use may further comprise buffers, solubilizers (such as cyclodextrins, ionic or non ionic surfactants, phospholipidic micellae or similar, microsomes or others), gelificants (such as Hyaluronic acid, hidroxymethyl-cellulose, hidroxypropyl-cellulose, carboxymethylcellulose, Xantan gum, tamarind seed polysaccharide, povidone, carbopol and/or others) and preservatives (such as Benzalkonium chloride, benzoxonium chloride, cetylpyridinium, polyquad and/or others).

According to the invention the above composition can be a collyrium or a solution suitable for imbibition of a contact lens, or (with appropriate buffers) suitable for ocular iontophoresis or for the inclusion in intraocular, fornix or intrapunctal porous solid insert (biodegradable or not) like polylactate or similar polimers. Preferably, according to the invention the formulation can be sterile eyedrops with or without gelificant(s); a sterile solution suitable for contact lens impregnation; a sterile solution suitable for transscleral iontophoresis; a concentrated solution for impregnation of a solid porous device to be placed in the inferior conjunctival fornix for a sustained release.

According to the invention preferably the antihistaminic/antiallergic principle includes but is not limited to: cromoglicic acid, Ketotifen, pemirolast, bepotastine besilate, aminophylline, astemizole, brompheniramine, carbinoxamine, cetirizine, chlorpheniramine, clemastine, diphenhydramine, doxylamine, ebastine, embramine, fexofenadine, levocetirizine, loratadine, methdilazine, mizolastine, rupatadine, terfenadine, quercetin, rutine, rosmarinic acid, caffeic acid esters, palmitoylethanolamide (PEA), luteolin, Perilla leaf extract, and Lindera obtusiloba water extract.

According to the invention preferably the anticholinergic principle includes but is not limited to: atropine base or salts thereof, hyoscyamine base or salts thereof, atropine methonitrate; anisotropine methylbromide, cyclopentolate, homatropine, 8-phenylacetyl homatropinium chloride, scopolamine (hyoscine), norscopolamine, metylscopolamine base or salts thereof, butylscopolamine base or salts thereof, ipratropium base or salts thereof, tiotropium base or salts thereof, oxitropium base or salts thereof, flutropium base or salts thereof, oxyphenonium base or salts thereof, cyclotropium base or salts thereof, cimetropium base or salts thereof, trospium base or salts thereof, xenytropium base or salts thereof, aclidinium base or salts thereof, clidinium base or salts thereof, tropicamide, cycrimine, biperiden, tolterodine, racanisodamine, ethopropazine, solifenacin, darifenacin, mebeverine, procyclidine, propantheline base or salts thereof, glycopyrrolate, isopropamide base or salts thereof, mepenzolate, tridihexethyl, hexocyclium methylsulfate, methoctramine, dicyclomine, flavoxate, oxybutynin, himbacine and analogs (see, e.g., WO 2005/118576; and WO 2006/076564), difenidol (Hexahydro-sila-difenidol, p-fluoro hexahydro-sila-difenidol), pirenzepine, telenzepine, nuvenzepine, rispenzepine and extract of the plants included in solanaceae family in particular the ones included in the tribes: Datureae, Hyoscyameae, Mandragoreae, Solandreae, Solaneae.

According to the invention preferably the dopaminergic principle, being mainly active on D2 receptors or as Dopamine reuptake inhibitor, includes but is not limited to: apomorphine, R(−)n-propylnorapomorphine, lergotrile, cabergoline, bromocryptine, 2-bromo-α-ergocryptine, dihydroergocryptine, pergolide, lisuride, levodopa, 3,4-dibenzoyl dopamine, dipropyldopamine, N-Methyldopamine, 3,4-dihydroxyphenylacetic acid (DOPAC), quinpirole, 7,8-Dihydroxy-5-phenyl-octahydrobenzo[h]isoquinoline, A-86929, dihydrexidine, dinapsoline, rotigotin, dinoxiline, doxanthrine, SKF81297, SKF-82958, SKF-38393, Fenoldopam, 6-Br-APB, A-68930, A-77636, CY-208,243, SKF-89145, SKF-89626, N, N-Propyldihydrexidine, Talipexole, Piribedil, Pramipexole, Quinelorane, Ropinirole, Sumanirole, cocaine, amphetamines, amantadine rimantadine and adapromine, Amineptine, bromantane, methylphenidate, dexmethylphenidate, difemetorex, fencamfamine, levophacetoperane, medifoxamine, mesocarb, nomifensine, pipradrol, prolintane, pyrovalerone,

According to the invention preferably the dopaminergic principle, being a dopamine antagonists mainly active on D1 receptors, includes but is not limited to: domperidone, metoclopramide, sulpiride, haloperidol, bulbocapnine, spiroperidol, thioproperazine, fluphenazine, pimozide, spiperone, SCH-23,390, SKF-83,959, Ecopipam (SCH-39,166), Eticlopride, Fallypride, Desmethoxyfallypride, L-741,626 (3-[4-(4-Chlorophenyl)-4-hydroxypiperidin-1-yl]methyl-1H-indole), Raclopride, Hydroxyzine, Itopride, SV 293, drugs classified as typical or atypical antipsychotics and Yohimbine.

According to the invention preferably the principle having a combined activity as anticholinergic and antihistaminic, or as anticholinergic, antihistaminic and dopaminergic (also as DA reuptake inhibitor) can be selected among Antiparkinsonian agents and tricyclic antidepressants thus includes but is not limited to: Benztropine base or salts thereof as Benztropine methanesulfonate (mesylate), etybenzatropine, Trihexyphenidyl, Ditran (JB-329) (70% 1-ethyl-2-pyrrolidinylmethyl-alpha-phenylcyclopentylglycolate and 30% 1-ethyl-3-piperidyl-alpha-phenylcyclopentylglycolate), 1-ethyl-3-piperidyl-alpha-phenylcyclopentylglycolate, methantheline, diphenylpyraline, ketamine, pethidine, tripelennamine, Dimenhydrinate, imipramine and metabolites thereof, amitriptyline and metabolites thereof, nortriptyline, 10-hydroxynortriptyline and desipramine.

A preferred combination for use according to the invention comprises atropine and Perilla leaf extract or, atropine and Ketotifen fumarate.

A preferred antiparkinsonian principle for use according to the invention is Benztropine mesylate.

Preferred buffers are Sodium phosphate monobasic and Sodium phosphate dibasic. Preferred preservative is Benzalkonium chloride.

A preferred ophthalmic formulation according to the invention comprises:

| | |
|---|---|
| atropine sulphate | 0.01%-1.0%, |
| Perilla leaf extract | 0.01%-5%, |
| Sodium phosphate monobasic | 0.05M, |
| Sodium phosphate dibasic | 0.05M, |
| Benzalkonium chloride | 0.025%-0.1%, |
| purified water | as remainder |

wherein the percentages are based on the total weight of the composition.

Another preferred ophthalmic formulation according to the invention comprises:

| | |
|---|---|
| atropine sulphate | 0.01%-1.0%, |
| Ketotifen fumarate | 0.01%-0.1% |
| Sodium phosphate monobasic | 0.05M, |
| Sodium phosphate dibasic | 0.05M, |
| Benzalkonium chloride | 0.025%-0.1%, |
| purified water | as remainder |

wherein the percentages are based on the total weight of the composition.

Another preferred ophthalmic formulation according to the invention comprises:

| | |
|---|---|
| benztropine mesylate | 0.001%-3.0% |
| Sodium phosphate monobasic | 0.05M, |
| Sodium phosphate dibasic | 0.05M, |
| Benzalkonium chloride | 0.025%-0.1%, |
| purified water | as remainder |

wherein the percentages are based on the total weight of the composition.

1-11. (canceled)

12. A method for stopping axial myopia in a subject in need thereof,
said method comprising administering topically or locally to the eye a single active pharmaceutical ingredient (API) having an antiparkinson activity wherein the single API is 0.001% to 3.0% Benztropine or a salt thereof.

13. The method according to claim 1 wherein the single API is 0.1% benztropine or a salt thereof.

14. The method according to claim 1 wherein the single API is Benztropine methansulfonate (mesylate).

15. The method according to claim 1 wherein the single API is administered in the form of an ophthalmic formulation selected in the group consisting of:
a sterile eye drops with or without gelificant(s);
a sterile solution suitable for contact lens impregnation;
a sterile solution suitable for transscleral iontophoresis; or
a concentrated solution for impregnation of a solid porous device to be placed in the inferior conjunctival fornix for a sustained release.

16. The method according to claim 4 wherein the ophthalmic formulation further comprises buffers, a solubilizer, gelificants and/or preservatives.

17. The method according to claim 5 wherein buffers are Sodium phosphate monobasic and Sodium phosphate dibasic and/or preservative is Benzalkonium chloride.

18. The method according to claim 4 wherein the ophthalmic formulation comprises:
0.1% Benztropine methanesulfonate (mesylate);
0.05 M Sodium phosphate monobasic;
0.05 M Sodium phosphate dibasic;
0.025% to 0.1% Benzalkonium chloride; and
purified water as remainder, wherein the percentages are based on the total weight of the composition.
